(19) United States
(12) Patent Application Publication
(10) Pub. No.: US 2024/0307145 A1
(43) Pub. Date: Sep. 19, 2024

(54) SURGICAL KIT AND METHODS OF FORMING THE SAME

(71) Applicant: NovoSource, LLC, Morgantown, WV (US)

(72) Inventors: Doug Hawkins, Morgantown, WV (US); Jacob Matheny, Westover, WV (US); Rob Brookover, Morgantown, WV (US); Joseph Jozwiak, Morgantown, WV (US)

(73) Assignee: NovoSource, LLC, Morgantown, WV (US)

(21) Appl. No.: 18/184,912

(22) Filed: Mar. 16, 2023

(51) Int. Cl. A61B 50/33; B33Y 80/00; B65B 5/02; B65B 5/08; B65D 77/00; B65D 77/04

(52) U.S. Cl. CPC A61B 50/33; B33Y 80/00; B65B 5/02; B65B 5/08; B65D 77/003; B65D 77/0406; A61B 2050/3007; B65D 2577/046

(57) ABSTRACT

A surgical kit includes a plurality of trays. Each of the plurality of trays includes plurality of surgical instruments made of a disposable material, with each of the plurality of surgical instruments being preassembled in the plurality of trays. The plurality of surgical instruments are arranged in the plurality of trays in an order in which the plurality of surgical instruments are to be used in a surgical procedure, and the plurality of trays are arranged in the surgical kit in the order in which the plurality of trays are to be used in the surgical procedure.

### TECHNICAL FIELD

The present specification generally relates to surgical kits, and more specifically, to surgical kits having preassembled surgical instruments.

### BACKGROUND

Traditionally, surgical procedures, such as knee replacement surgeries, are performed with numerous instruments that are delivered to a surgeon and/or technician in a plurality of trays. The instruments that are packaged in the trays are often broken down into a plurality of components in order to aid packaging in the plurality of trays. As a result, the surgeon and/or technician performing the procedure may be responsible for assembling the instruments prior to and/or during the procedure. Given the complexity of these instruments and the operations which they are used to perform, assembling instruments during a procedure may lead to surgical inefficiencies and errors. Further, excessive handling of the instruments for the purposes of assembly may increase the chances of contamination of the instruments.

### SUMMARY

In an embodiment, a surgical kit is disclosed. The surgical kit includes a plurality of trays. Each of the plurality of trays includes plurality of surgical instruments made of a disposable material, with each of the plurality of surgical instruments being preassembled in the plurality of trays. The plurality of surgical instruments are arranged in the plurality of trays in an order in which the plurality of surgical instruments are to be used in a surgical procedure, and the plurality of trays are arranged in the surgical kit in the order in which the plurality of trays are to be used in the surgical procedure.

In another embodiment, a surgical kit for performing a total knee replacement procedure is disclosed. The surgical kit includes a first tray, a second tray, and a third tray. The first tray includes a first plurality of preassembled surgical instruments of a first type that are used to perform a first step of a total knee replacement procedure. The second tray includes a second plurality of preassembled surgical instruments of a second type that are used to perform a second step of a total knee replacement procedure. The third tray includes a third plurality of surgical instruments of a third type that are used to perform a third step of a total knee replacement surgery. The first plurality of preassembled surgical instruments of the first type, the second plurality of preassembled surgical instruments of the second type, and the third plurality of preassembled surgical instruments of the third type are arranged in an order in which they are to be used during the total knee replacement procedure.

In yet another embodiment, a method of forming a surgical kit is disclosed. The method may involve forming a plurality of trays, and the plurality of trays may include a plurality of cavities that receive a plurality of surgical instruments. The method may further include forming the plurality of surgical instruments, and preassembling the plurality of surgical instruments. Once the plurality of trays and the plurality of surgical instruments are formed, the method may involve securing the plurality of preassembled surgical instruments in the plurality of cavities of the plurality of trays. The method may then involve wrapping each of the plurality of trays in a sterile material and arranging the plurality of trays in an order in which the plurality of trays are to be used during a surgical procedure. The method may finally involve wrapping the surgical kit in a sterile material.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 depicts a perspective view of an illustrative surgical kit, according to one or more embodiments shown and described herein;

FIG. 2 depicts a schematic top-down view of an illustrative first tray of a surgical kit, according to one or more embodiments shown and described herein;

FIG. 3 depicts a schematic top-down view of an illustrative second tray of a surgical kit, according to one or more embodiments shown and described herein;

FIG. 4 depicts a schematic top-down view of an illustrative third tray of a surgical kit, according to one or more embodiments shown and described herein; and

FIG. 5 depicts a flow diagram of an illustrative method of forming a surgical kit, according to one or more embodiments shown and described herein.

### DETAILED DESCRIPTION

Embodiments disclosed herein relate to surgical kits and methods of forming surgical kits, in particular knee replacement kits for total knee replacement procedures. More specifically, the present disclosure includes a surgical kit having a plurality of trays, where each of the plurality of trays further includes a plurality of disposable, pre-assembled surgical instruments. In these embodiments, the surgical instruments may be packaged within the plurality of trays in the order in which the surgical instruments are to be used during a surgical procedure. Accordingly, the surgical kits and methods described herein allow for use of components in a manner that minimizes or reduces surgical inefficiencies and errors by avoiding and/or minimizing pre-surgical handling, pre-surgical assembly procedures, excessive contact with components that may result in contamination, and/or the like.

As provided herein, the term “total knee replacement” may refer to a surgical process by which damaged cartilage and bone are removed from a knee joint and replaced by a joint.

As provided herein, the term “wrapped” may refer to the process of enclosing a surgical kit or surgical tray in a sterile material, including containers, peel pouches of plastic and/or paper, or sterilization wraps.

As provided herein, the term “3D-printed” may refer to the additive manufacturing process used to create a desired object, such as a surgical instrument, by stacking (e.g., printing) a plurality of layers of plastic, resin, metal, or the like, to create a solid three-dimensional part. The 3D-printing process may involve generating a digital model, such as a computer-aided design (CAD) model, which may include a plurality of horizontal layers to create a template for the 3D printer device to follow.

As described in more detail herein, traditional surgical procedures are performed using surgical kits having numerous instruments that are delivered in a plurality of trays. Furthermore, the numerous instruments in each tray are often broken down into a plurality of unassembled components, thereby requiring that a surgeon and/or technician take on the time consuming task of assembling the instruments prior to and/or during a surgical procedure. Given the complexity of many surgical instruments, requiring a surgeon and/or technician to assemble the instruments may result in inefficiencies and/or errors during a procedure, may prolong a procedure (and the amount of time a subject is under anesthesia), may result in the wrong instruments being used for a particular phase of a procedure, and/or the like. These issues may be exacerbated in instances where a surgeon is working with a technician who is inexperienced with the particular procedure being performed.

To further complicate matters, standard surgical kits may include upwards of ten trays, with each tray weighing up to 40 pounds (e.g., up to about 18 kg). As a result, these surgical kits are often difficult to maneuver in particular settings (e.g., in a hospital or other medical facility). Furthermore, a surgeon and/or technician may have difficulty identifying a requisite instrument from the plurality of trays, which can lead to untimely delays during a surgical procedure.

Because these surgical kits are often reused, a facility may also collect each of the surgical instruments for sterilization following each surgical procedure. However, due to the nature of traditional reusable surgical instruments, blood, bone, or tissue that becomes lodged in an instrument during a procedure may not be removable from the instrument, and thus the instrument cannot be cleaned and/or sterilized for subsequent use. In these instances, reuse of a contaminated instrument could cause subsequent subjects to experience infections that may ultimately lead to bone loss, reduced function in the surgically repaired appendage, significant pain, and/or other negative long term effects.

Furthermore, a facility may run the risk of losing particular instruments in the process of collecting the surgical kits for sterilization. Oftentimes, surgical kits may include hundreds of unassembled components, with many of the components including small pins and/or screws that may be easily misplaced. In the event a component (or an entire instrument) is misplaced post-procedure, a surgeon and/or technician may find that the repackaged surgical kit is missing the requisite components during a subsequent procedure.

Even in instances when the surgical kits are sterilized and repackaged without the loss of any components, surgeons and/or technicians may find that the performance of certain instruments may suffer after a single (or a plurality of) use(s). For example, instruments may be damaged (e.g., bent, chipped, burred, etc.) during a procedure, while certain cutting instruments may dull or otherwise become worn down over repeated uses. As can be appreciated, any damage to the instrument in the surgical kit may result in complications in subsequent procedures.

It should also be noted that facilities incur significant costs to reutilize surgical kits across a plurality of procedures. For example, in order to reuse a single surgical kit, a facility gathers each of the instruments post-procedure, sterilizes the instruments, repackages the instruments, and wraps the surgical kit such that the kit remains sterile until it is utilized for a subsequent procedure. Traditional surgical kits may cost upwards of 700 U.S. dollars or more to prepare for reuse.

Accordingly, a need exists for a surgical kit that may help remedy the deficiencies of traditional surgical kits noted herein. More particularly, a need exists for a disposable surgical kit that includes preassembled instruments that are packaged in the order in which they are utilized during a procedure. It should be noted that having a disposable surgical kit effectively addresses issues with sanitation, sterilization, repackaging, and instrument damage, as a surgeon and/or technician may utilize a new surgical kit for each procedure. Furthermore, by preassembling the surgical instruments within the surgical kit, many of the shortcomings related to assembling the instruments during a surgical procedure may be alleviated.

Embodiments of surgical kits and methods for forming surgical kits will now be described in more detail herein. The following will now describe these surgical kits and methods in more detail with reference to the drawings and where like numbers refer to like structures.

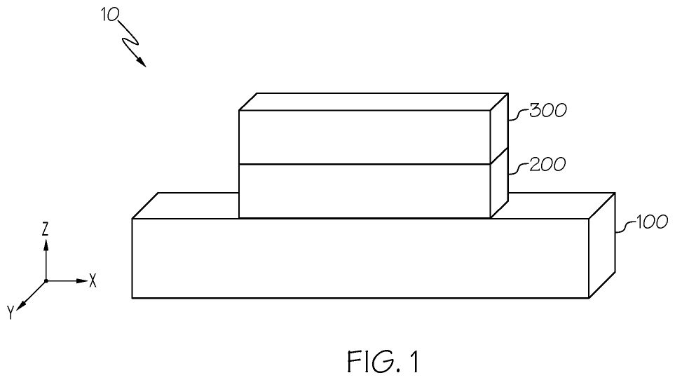

Referring now to FIG. 1, a surgical kit 10 is depicted. In these embodiments, the surgical kit 10 may be a surgical kit configured for performing a total knee replacement surgery or similar procedure. However, it should be understood that the surgical kit 10 may be utilized for any requisite surgical procedure without departing from the scope of the present disclosure.

As illustrated in FIG. 1, the surgical kit 10 may include a plurality of trays, such as a first tray 100, a second tray 200, and a third tray 300. Although the surgical kit 10 is depicted as having three trays 100, 200, 300, it should be noted that the surgical kit 10 may include any number of trays necessary to perform a particular procedure, such as two trays, four trays, five trays, eight trays, ten trays, or any other number of trays. In these embodiments, the plurality of trays 100, 200, 300 may be formed of a disposable material, such as thermoplastics including, but not limited to, polyethylene terephthalate (PET), amorphous polyethylene terephthalate (APET), polyethylene terephthalate glycol-modified (PETG), and high impact polystyrene (HIPS). Accordingly, each of the plurality of trays may be disposed of following a surgical procedure. In some embodiments, the plurality of trays 100, 200, 300 may be 3D-printed using an additive manufacturing process, while in other embodiments, the plurality of trays 100, 200, 300 may be formed using an injection-molding process.

The plurality of trays 100, 200, 300 may be wrapped together in a stacked configuration, as is depicted in FIG. 1. In these embodiments, the plurality of trays 100, 200, 300, may be arranged in a stack based on their order of use during a particular surgical procedure. For example, first tray 100 may include surgical instruments utilized during a first step of a surgical procedure, second tray 200 may include surgical instruments utilized during a second step of a surgical procedure, and third tray 300 may include surgical instruments utilized during a third step of a surgical procedure. As a result, the first tray 100 may be positioned at the bottom of the stack (e.g., in the −z direction relative the second tray 200 and third tray 300, as depicted in the coordinate axes of FIG. 1), the second tray 200 may be positioned in the middle of the stack (e.g., in the +z direction relative the first tray 100 and in the −z direction relative the third tray, as depicted in the coordinate axes of FIG. 1), and the third tray 300 may be positioned at the top of the stack (e.g., in the +z direction relative the second tray 200, as depicted in the coordinate axes of FIG. 1) of the stack.

As a result, when a surgeon and/or technician unwraps the surgical kit, the first tray 100 may be utilized to perform the first step in the surgical procedure. Once the first step of the surgical procedure is complete, the first tray 100 may be removed from the stack or discarded, and the second tray 200 may be used to perform the second step of the surgical procedure. Similarly, the second tray 200 may be removed from the stack or discarded following completion of the second surgical step, and the third tray 300 may be used to perform the third step of the surgical procedure.

It should be further noted that, although the disposable nature of the plurality of trays may allow for the trays to be discarded after use, in some embodiments, one or more of the plurality of trays may be recycled for use in additional procedures. For instance, in the event a particular tray is not utilized during a procedure, the tray may be repackaged in a new surgical kit and set aside for use in a subsequent procedure. Similarly, if one or more of the plurality of trays are not contaminated and/or damaged during a procedure, it may be possible to reutilize the one or more of the plurality of trays in subsequent procedures. As such, an advantage of the present systems is realized in that portions of the stack of the trays can be reused, as opposed to other trays where the whole stack must be discarded, regardless of whether certain components have been used.

Although not depicted, it should be understood that the surgical kit 10 may be wrapped prior to a procedure to ensure that the surgical kit is not contaminated. For example, the surgical kit may be wrapped in polyethylene terephthalate (PET), amorphous polyethylene terephthalate (APET), Polyethylene terephthalate glycol-modified (PETG), high impact polystyrene (HIPS) or any other similar material, with the wrapping of the surgical kit being removed immediately prior to a procedure. In some embodiments, the surgical kit 10 may be further wrapped in a blister pack that may be peeled off prior to a procedure. Furthermore, in addition to wrapping the surgical kit 10 as a whole, each of the plurality of trays 100, 200, 300 may be individually wrapped for extra protection. In these embodiments, each of the plurality of trays 100, 200, 300 may be unwrapped immediately prior to being utilized during a surgical procedure.

Referring still to FIG. 1, for exemplary purposes, the surgical kit 10 may be utilized to perform a total knee replacement surgery. Traditionally, once a treatment area has been accessed, total knee replacement procedures may involve a plurality of steps. For example, a total knee replacement procedure may include a first step involving preparing a femoral bone to receive a femoral component, and fitting the femoral component on the femoral bone. The procedure may further involve a second step involving preparing a tibial bone to receive a tibial component, and fitting the tibial component on the tibial bone. The procedure may also involve a third step, including adjusting a patellar bone to fit the femoral component and the tibial component, and finalizing the procedure.

In these embodiments, the first tray 100 may include a first plurality of instruments of a first type, such as femoral instruments, that may be used to perform the first step of the knee replacement procedure. Similarly, the second tray 200 may include a second plurality of instruments of a second type, such as tibial instruments, that may be used to perform the second step of the knee replacement procedure. The third tray 300 may include a third plurality of instruments of a third type, such as patellar instruments, that may be used to perform the third step of the knee replacement procedure. The plurality of trays 100, 200, and 300 will be described in additional detail herein with reference to the relevant figures.

It should be understood that the surgical kit 10 depicted in FIG. 1 is for exemplary purposes only, as the plurality of trays 100, 200, 300 may be arranged in any order and in any configuration without departing from the scope of the present disclosure. Furthermore, it should be understood that the first tray 100, the second tray 200, and the third tray 300 may each include any type of instruments without departing from the scope of the present disclosure. For example, particular surgeons and/or technicians may desire that the plurality of trays be arranged in a particular configuration, or that the first tray 100, the second tray 200, and the third tray 300 contain specific instruments. Accordingly, the surgical kit 10 depicted in FIG. 1 may be customized in order to conform to the preferences of the surgeon and/or technician utilizing the surgical kit.

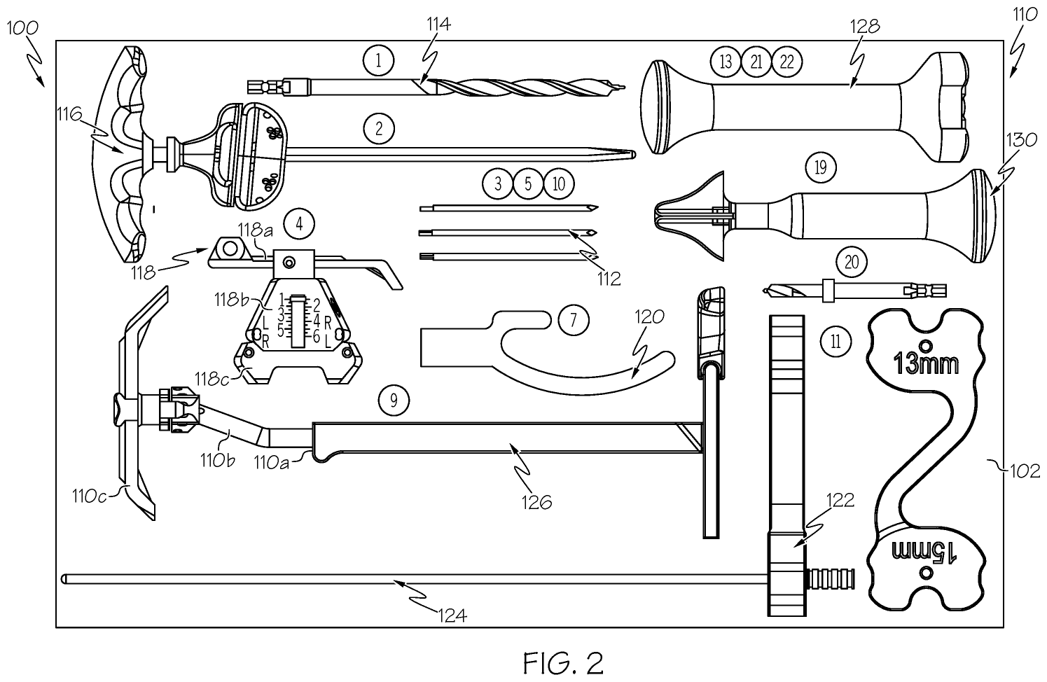

Referring now to FIG. 2, a top-side view of an illustrative first tray 100 is depicted. In these embodiments, the first tray 100 may include a first plurality of cavities 102 into which a first plurality of surgical instruments 110 are placed. In these embodiments, the first plurality of cavities 102 may be designed to fit the first plurality of surgical instruments 110, such that each of the first plurality of surgical instruments 110 fit snugly within its respective cavity 102.

Referring still to FIG. 2, each of the first plurality of surgical instruments 110 may be preassembled prior to being inserted into the first tray 100. In these embodiments, some of the plurality of surgical instruments 110 may include a plurality of components, such as components 110a, 110b, and 110c. Rather than packaging the plurality of components individually within the first tray 100, the components may be coupled together to form at least one of the first plurality of surgical instruments 110, with the assembled first surgical instrument 110 being packaged in the tray 100. In these embodiments, the first plurality of surgical instruments 110 may be of a first type, such that the first plurality of surgical instruments 110 corresponds with a first step of a surgical procedure.

For example, FIG. 2 illustrates an exemplary first tray 100 of a surgical kit 10 for performing a total knee replacement procedure. In these embodiments, the first plurality of surgical instruments 110 may include the following surgical instruments: a femoral drill 114, a femoral alignment block 116, a femoral sizing block 118, a blade gauge 120, an extension block 122, an alignment rod 124, a tibial cutting guide 126, an insert impactor 128, and a keel punch 130. Furthermore, it should be noted that the first tray 100 may include additional non-surgical instruments, such as a plurality of pins 112. In these embodiments, the plurality of pins 112 may aid in operation of at least one of the first plurality of surgical instruments (e.g., cutting guides, etc.).

As has been noted herein at least one of the first plurality of surgical instruments 110 may include a plurality of components. For example, as depicted in FIG. 2, the femoral sizing block 118 may include a first component 118a, such as a sizing block, a second component 118b, such as a stylus, and a third component 118c, such as feet, which may be each coupled together prior to being inserted into the first tray 100.

In some embodiments, the first plurality of surgical instruments 110 may be formed of a disposable material, such as such as nylon, glass filled nylon, stainless steel, aluminum, acetal copolymer, or other thermoplastics including, but not limited to, polycarbonate, polypropylene, polyethylene, polyphenylsulfone. As a result, each of the first plurality of surgical instruments 110 may be disposed following a procedure. Furthermore, it should be noted that, in some embodiments, the first plurality of surgical instruments 110 may be 3D-printed. In these embodiments, any of the first plurality of surgical instruments 110 which include the plurality of components 110a, 110b, 110c may be 3D-printed as a monolithic structure, which may alleviate the need to assemble the plurality of components 110a, 110b. 110c prior to packaging.

For example, in some embodiments, the first tray 100 and each of the first plurality of surgical instruments 110 may be 3D-printed. In these embodiments, a 3D-printer may be provided a set of print instructions corresponding to the formation of the first tray 100 and the plurality of surgical instruments 110, such that each of the first tray 100 and the plurality of surgical instruments may be formed simultaneously.

In other embodiments, the first tray 100 may be formed from a non-additive manufacturing process (e.g., injection molding, etc.) and placed in a 3D-printer. Once the first tray 100 has been aligned in the 3D-printer, the first plurality of surgical instruments 110 may be printed directly into the cavities 102 formed in the first tray 100. In these embodiments, any of the first plurality of surgical instruments 110 that include the plurality of components 110a. 110b, 110c may be printed as single, monolithic structures within the first tray 100.

In other embodiments still, each of the plurality of components 110a, 110b, 110c of the first plurality of surgical instruments 110 may be 3D-printed. In these embodiments, once the plurality of components 110a, 110b, 110c are formed, the plurality of components 110a, 110b, 110c may be manually assembled to form the first plurality of surgical instruments 110 prior to placing the preassembled instruments in the first tray 100.

Referring still to FIG. 2, the first plurality of surgical instruments 110 may further be of the first type. In these embodiments, the first plurality of surgical instruments 110 may be used to perform the first step of a surgical procedure, such as a total knee replacement procedure. In order to indicate that the first plurality of surgical instruments 110 may be intended for the first step of the surgical procedure, the first tray 100 may include a variety of indicia that indicates the order in which each of the surgical instruments is to be used during a procedure. For example, as depicted in FIG. 2, the first tray 100 includes a set of numbers next to the cavities 102 of each the plurality of surgical instruments 110. These indicia may represent the order in which each of the first plurality of surgical instruments 110 are utilized during the first step of the surgical procedure.

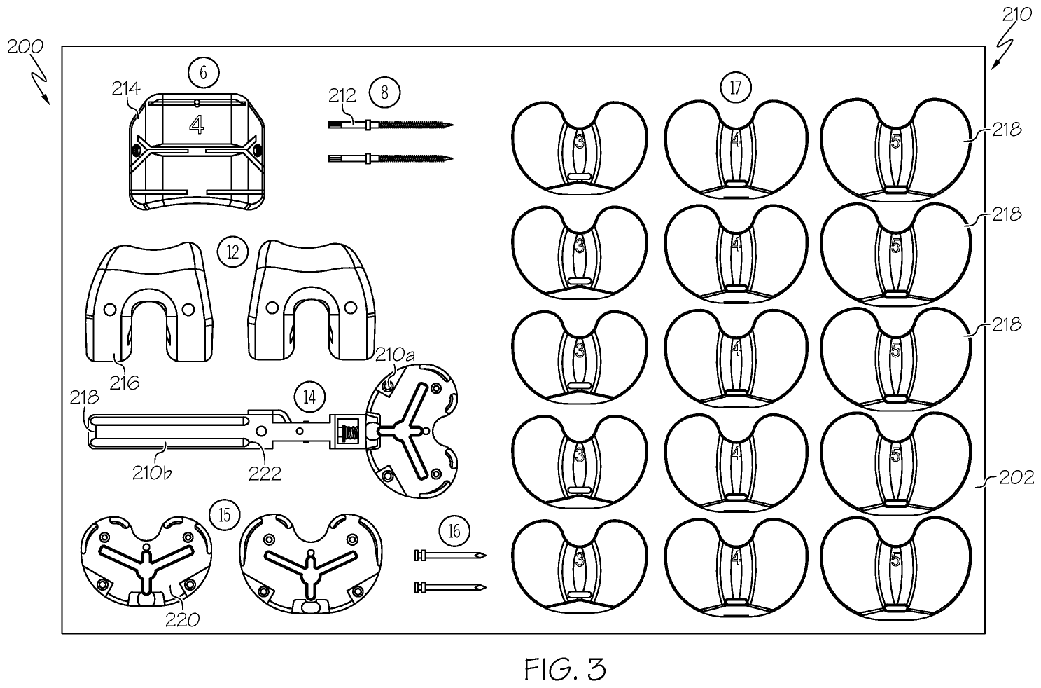

Referring now to FIG. 3, a top-side view of an illustrative second tray 200 is depicted. In these embodiments, the second tray 200 may include a second plurality of cavities 202 into which a second plurality of surgical instruments 210 are placed. In these embodiments, the second plurality of cavities 202 may be designed to fit the second plurality of surgical instruments 210, such that each of the second plurality of surgical instruments 210 fit snugly within its respective cavity 202.

Referring still to FIG. 3, each of the second plurality of surgical instruments 210 may be preassembled prior to being inserted into the second tray 200. In these embodiments, some of the plurality of surgical instruments 210 may include a plurality of components 210a. 210b. Rather than packaging the plurality of components 210a, 210b individually within the second tray 200, the components may be coupled together to form at least one of the second plurality of surgical instruments 210, with the assembled second surgical instrument 210 being packaged in the tray 200. In these embodiments, the second plurality of surgical instruments 210 may be of the second type, such that the second plurality of surgical instruments 210 corresponds with a second step of a surgical procedure.

For example, FIG. 3 illustrates an exemplary second tray 200 of a surgical kit 10 for performing a total knee replacement procedure. In these embodiments, the second plurality of surgical instruments 210 may include the following surgical instruments: a femoral cutting block 214, a femoral trial 216, a tibial tray trial 218, a tibial tray holder 220, and an insert trial 222. Furthermore, it should be noted that the second tray 200 may include additional non-surgical instruments, such as a plurality of pins 212. In these embodiments, the plurality of pins 212 may aid in operation of at least one of the second plurality of surgical instruments (e.g., keel guides, etc.).

As has been noted herein at least one of the second plurality of surgical instruments 210 may include a plurality of components 210a, 210b. For example, as depicted in FIG. 3, the tibial tray holder may include a first component 210a, such tibial tray holder 220, and a second component 210b, such as a keel guide 224, which may be coupled together prior to being inserted into the second tray 200.

In some embodiments, the second plurality of surgical instruments 210 may be formed of a disposable material, such as such as nylon, glass filled nylon, stainless steel, aluminum, acetal copolymer, or other thermoplastics including, but not limited to, polycarbonate, polypropylene, polyethylene, polyphenylsulfone. As a result, each of the second plurality of surgical instruments 210 may be disposed of following a procedure. Furthermore, it should be noted that, in some embodiments, the second plurality of surgical instruments 210 may be 3D-printed. In these embodiments, any of the second plurality of surgical instruments 210 which include the plurality of components may be 3D-printed as a monolithic structure, which may alleviate the need to assemble the plurality of components prior to packaging.

For example, in some embodiments, the second tray 200 and each of the second plurality of surgical instruments 210 may be 3D-printed. In these embodiments, a 3D-printer may be provided a set of print instructions corresponding to the formation of the second tray 200 and the plurality of surgical instruments 210, such that each of the second tray 200 and the plurality of surgical instruments may be formed simultaneously.

In other embodiments, the second tray 200 may be formed from a non-additive manufacturing process (e.g., injection molding, etc.) and placed in a 3D-printer. Once the second tray 200 has been aligned in the 3D-printer, the second plurality of surgical instruments 210 may be printed directly into the cavities 202 formed in the second tray 200. In these embodiments, any of the second plurality of surgical instruments 210 that include the plurality of components 210a. 210b, 210c may be printed as single, monolithic structures within the second tray 200.

In other embodiments still, each of the plurality of components 210a, 210b, 210c of the second plurality of surgical instruments 210 may be 3D-printed. In these embodiments, once the plurality of components 210a. 210b, 210c are formed, the plurality of components 210a. 210b, 210c may be manually assembled to form the second plurality of surgical instruments 210 prior to placing the preassembled instruments in the second tray 200.

Referring still to FIG. 3, the second plurality of surgical instruments 210 may further be of the second type. In these embodiments, the second plurality of surgical instruments 210 may be used to perform the second step of a surgical procedure, such as a total knee replacement procedure. In order to indicate that the second plurality of surgical instruments 210 may be intended for the second step of the surgical procedure, the second tray 200 may include a variety of indicia that indicates the order in which each of the surgical instruments is to be used during a procedure. For example, as depicted in FIG. 3, the second tray 200 includes a set of numbers next to the cavities 202 of each the plurality of surgical instruments 210. These indicia may represent the order in which each of the second plurality of surgical instruments 210 are utilized during the second step of the surgical procedure.

Referring now to FIGS. 2 and 3, it should be noted that the first plurality of surgical instruments 110 may be different from the second plurality of surgical instruments 210. For example, in some embodiments, each of the first plurality of surgical instruments 110 in the first tray 100 may be of the first type, while the second plurality of surgical instruments 210 in the second tray 200 may be of the second type. In these embodiments, the first type may be different from the second type, such that each of the first plurality of surgical instruments 110 may be different from each of the second plurality of surgical instruments 210. As such, in some embodiments, the first tray 100 and the second tray 200 may contain none of the same surgical instruments. Additionally, in these embodiments, the second plurality of surgical instruments may be used to perform the second step of a surgical procedure, such as a total knee replacement procedure.

Furthermore, in some embodiments, a second size of the second plurality of surgical instruments 210 in the second tray 200 may be determined based on a first size of the first plurality of surgical instruments 110 in the first tray 100. For example, the first plurality of surgical instruments 210 may include a femoral sizing block of a first size, where the first size corresponds to the needs of a subject undergoing a total knee replacement procedure. In these embodiments, the second size of at least one of the second plurality of surgical instruments 210 in the second tray 200 may correspond to the at least one first plurality of surgical instruments 110 having the first size. For example, the second tray 200 may include a tibial tray holder and/or keel guide having the second size, and the second size of the tibial tray holder and/or keel guide may correspond to the first size of the femoral block, such that the surgical instruments may be appropriately utilized during a procedure.

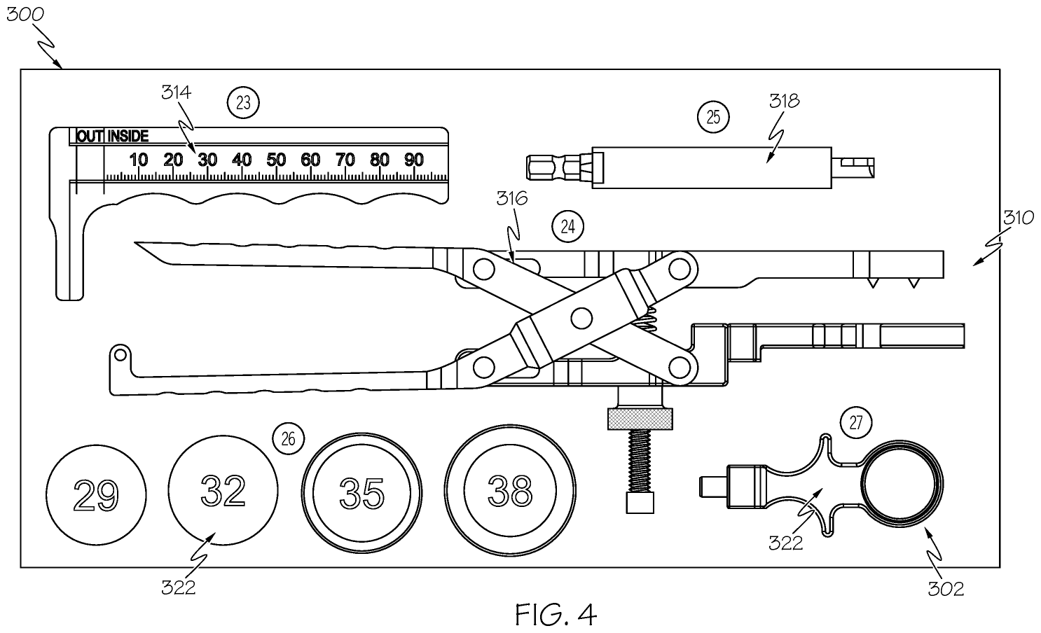

Referring now to FIG. 4, a top-side view of an illustrative third tray 300 is depicted. In these embodiments, the third tray 300 may include a third plurality of cavities 302 into which a third plurality of surgical instruments 310 are placed. In these embodiments, the third plurality of cavities 302 may be designed to fit the third plurality of surgical instruments 310, such that each of the third plurality of surgical instruments 310 fit snugly within its respective cavity 302. Although the surgical kit 10 is depicted as having the third tray 300, it should be understood that, in some embodiments, the third tray 300 may be optional. For example, in some embodiments, the surgical procedure may be conducted using only the first tray and the second tray, along with the corresponding surgical instruments.

Referring still to FIG. 4, each of the third plurality of surgical instruments 310 may be preassembled prior to being inserted into the third tray 300. In these embodiments, some of the plurality of surgical instruments 310 may include a plurality of components. Rather than packaging the plurality of components individually within the third tray 300, the components may be coupled together to form at least one of the third plurality of surgical instruments 310, with the assembled third surgical instrument 310 being packaged in the tray 300. In these embodiments, the third plurality of surgical instruments 310 may be of a third type, such that the third plurality of surgical instruments 310 corresponds with a third step of a surgical procedure.

For example, FIG. 4 illustrates an exemplary third tray 300 of a surgical kit 10 for performing a total knee replacement procedure. In these embodiments, the third plurality of surgical instruments 310 may include the following surgical instruments: a patella caliper 314, a patellar drill guide 316, a patellar drill 318, a patellar trial 320, and a patellar clamp 322. Furthermore, it should be noted that the third tray 300 may include additional non-surgical instruments, such as a plurality of pins. In these embodiments, the plurality of pins may aid in operation of at least one of the third plurality of surgical instruments.

In some embodiments, the third plurality of surgical instruments 310 may be formed of a disposable material, such as nylon, glass filled nylon, stainless steel, aluminum, acetal copolymer, or other thermoplastics including, but not limited to, polycarbonate, polypropylene, polyethylene, polyphenylsulfone. As a result, each of the third plurality of surgical instruments 310 may be disposed of following a procedure. Furthermore, it should be noted that, in some embodiments, the third plurality of surgical instruments 310 may be 3D-printed. In these embodiments, any of the third plurality of surgical instruments 310 which include the plurality of components may be 3D-printed as a monolithic structure, which may alleviate the need to assemble the plurality of components prior to packaging. In some embodiments, the various components that are 3D printed may include frangible portions that are separable from one another during a procedure.

For example, in some embodiments, the third tray 300 and each of the third plurality of surgical instruments 310 may be 3D-printed. In these embodiments, a 3D-printer may be provided a set of print instructions corresponding to the formation of the third tray 300 and the plurality of surgical instruments 310, such that each of the third tray 300 and the plurality of surgical instruments may be formed simultaneously.

In other embodiments, the third tray 300 may be formed from a non-additive manufacturing process (e.g., injection molding, etc.) and placed in a 3D-printer. Once the third tray 300 has been aligned in the 3D-printer, the third plurality of surgical instruments 310 may be printed directly into the cavities 302 formed in the third tray 300. In these embodiments, any of the third plurality of surgical instruments 310 that include a plurality of components may be printed as single, monolithic structures within the third tray 300.

In other embodiments still, each of the plurality of components of the third plurality of surgical instruments 310 may be 3D-printed. In these embodiments, once the plurality of components are formed, the plurality of components may be manually assembled to form the third plurality of surgical instruments 310 prior to placing the preassembled instruments in the third tray 300.

Referring still to FIG. 4, the third plurality of surgical instruments 310 may further be of the third type. In these embodiments, the third plurality of surgical instruments 310 may be used to perform the third step of a surgical procedure, such as a total knee replacement procedure. In order to indicate that the third plurality of surgical instruments 310 may be intended for the third step of the surgical procedure, the third tray 300 may include a variety of indicia that indicates the order in which each of the surgical instruments is to be used during a procedure. For example, as depicted in FIG. 4, the third tray 300 includes a set of numbers next to the cavities 302 of each the plurality of surgical instruments 310. These indicia may represent the order in which each of the third plurality of surgical instruments 310 are utilized during the third step of the surgical procedure.

Referring now to FIGS. 3-4, it should be noted that the third plurality of surgical instruments 310 may be different from both the first plurality of surgical instruments 110 and the second plurality of surgical instruments 310. For example, in some embodiments, each of the third plurality of surgical instruments 310 in the third tray 300 may have the third type while the second plurality of surgical instruments 210 in the second tray 200 may be of the second type and the first plurality of surgical instruments 110 in the first tray 100 may be of the first type. In these embodiments, the third type may be different from both the second type and the first type, such that the first tray 100, the second tray 200, and the third tray 300 contain none of the same surgical instruments.

It should be noted that the examples provided herein with reference to FIGS. 3-4 are intended for illustrative purposes only. For example, it should be understood that the first tray 100, the second tray 200, and third tray 300 may include any variation of surgical instruments without departing from the scope of the present disclosure. Furthermore, the plurality of trays 100, 200, and 300 and the plurality of surgical instruments 110, 210, 310 may be arranged in any order that may be desired by a particular surgeon and/or technician and/or for a particular surgical procedure.

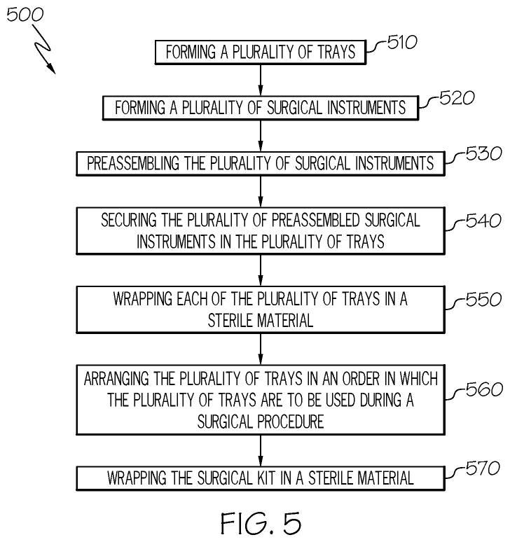

Turning now to FIG. 5, an illustrative method 500 of forming a surgical kit is depicted. As shown at block 510, the method may first involve forming a plurality of trays. In these embodiments, the plurality of trays may be formed to include a plurality of cavities, which may be operable to receive a plurality of surgical instruments. In some embodiments, the plurality of trays may be formed using an additive manufacturing process, such as 3D-printing. In these embodiments, the each of the plurality of trays may be formed as a single, monolithic structure, with the plurality of cavities being 3D-printed into the plurality of trays.

In some embodiments, forming the plurality of trays may involve forming a first tray, a second tray, and a third tray, although it should be noted that any number of trays may be formed without departing from the scope of the present disclosure. In these embodiments, each of the first tray, the second tray, and the third tray may include a plurality of cavities. The plurality of cavities in each of the first tray, the second tray, and the third tray may have varying designs (e.g., size, shape, etc.), such that each of the first tray, the second tray, and the third tray may be operable to receive different surgical instruments.

Once the plurality of surgical trays are formed, the method may move to block 520, which may involve forming the plurality of surgical instruments. In these embodiments, the plurality of surgical instruments may also be formed using an additive manufacturing process, such as 3D-printing. In these embodiments, each of the plurality of surgical instruments may be formed as single, monolithic structure, and may further be formed to fit into at least one of the corresponding plurality of cavities formed in the plurality of trays.

As depicted at block 530, the method may further involve preassembling the plurality of surgical instruments. In these embodiments, at least one of the plurality of surgical instruments may include a plurality of components. Thus, the plurality of components may be coupled together prior to placing the at least one of the plurality of surgical instruments into its respective one of the plurality of cavities formed in the plurality of trays.

With the surgical instruments formed, the method may proceed to block 540, which may involve securing the plurality of preassembled surgical instruments in the plurality of cavities of the plurality trays of the surgical kit. In some embodiments, the plurality of cavities may be designed to receive a specific surgical instrument of the plurality of surgical instruments, such that the surgical instrument fits snugly within its respective cavity. In other embodiments, the plurality of cavities may have a general shape (e.g., square, rectangular, circular etc.) such that the plurality of surgical instruments may be placed within the plurality of cavities of the plurality of trays.

Moving now to block 550, the method 500 may further involve wrapping each of the plurality of trays in a sterile material. In these embodiments, the step of wrapping the plurality of trays may further aid in securing the plurality of surgical instruments in the plurality of cavities of the plurality of trays. Furthermore, the sterile wrapping may ensure that the surgical instruments are not contaminated prior to being opened and used to perform a surgical procedure.

With each of the plurality of trays wrapped, the plurality of trays may be arranged in the order in which the plurality of trays are to be used during a surgical procedure, as shown at block 560. For example, in embodiments in which the plurality of trays include a first tray, a second tray, and a third tray, each of the trays may be arranged in a stacked configuration. In these embodiments, the first tray may be utilized during a first step of the procedure, and may be positioned on the top of the stacked configuration. The second tray may be utilized during a second step of the procedure, and may be positioned between the first tray and the third tray. The third tray may be utilized during a third step of the procedure, and may be positioned beneath the second tray.

Once the plurality of trays are appropriately arranged, each of the plurality of trays forming the surgical kit may be wrapped together in a sterile material, as shown at block 570. By wrapping the plurality of trays together, it is possible to ensure that the plurality of trays remain in the desired arrangement. Furthermore, the additional sterile wrapping layer aids in preventing contamination of the plurality of trays and the plurality of surgical instruments prior to the surgical kit being opened to perform a surgical procedure.

As should be appreciated in view of the foregoing, a surgical kit is described herein. The surgical kit may include a plurality of trays, with each of the plurality of trays having a plurality of surgical instruments made of a disposable material. Furthermore, the plurality of surgical instruments may be preassembled in the plurality of trays. In embodiments, the plurality of surgical instruments are arranged in the plurality of trays in an order in which the plurality of surgical instruments are to be used in a surgical procedure, and the plurality of trays are arranged in the surgical kit in the order in which the plurality of trays are to be used in the surgical procedure. By preassembling the surgical instruments within the surgical kit, and arranging the instruments in the order in which they are to be used during the procedure, many of the surgical inefficiencies and errors that arise from assembling instruments while the procedure is being performed may be alleviated.

Further aspects of the embodiments described herein are provided by the subject matter of the following items:

1. A surgical kit comprising: a plurality of trays, the plurality of trays comprising: a plurality of surgical instruments made of a disposable material, the plurality of surgical instruments being preassembled in the plurality of trays; wherein the plurality of surgical instruments are arranged in the plurality of trays in an order in which the plurality of surgical instruments are to be used in a surgical procedure, and the plurality of trays are arranged in the surgical kit in the order in which the plurality of trays are to be used in the surgical procedure.

2. The surgical kit of claim 1, wherein the surgical kit is wrapped in a sterile material.

3. The surgical kit of claim 1, wherein each of the plurality of trays are wrapped in a sterile material.

4. The surgical kit of claim 1, wherein the plurality of trays include at least a first tray and a second tray.

5. The surgical kit of claim 4, wherein the first tray includes a first plurality of surgical instruments of a first type to be used during a first step of the surgical procedure, and the second tray includes a second plurality of surgical instruments of a second type to be used during a second step of the surgical procedure.

6. The surgical kit of claim 5, wherein the plurality of trays further includes a third tray, and the third tray includes a third plurality of surgical instruments of a third type to be used during a third step of the surgical procedure.

7. The surgical kit of item 1, wherein the plurality of trays each include a plurality of cavities designed to receive the plurality of preassembled surgical instruments.

8. The surgical kit of item 1, wherein a plurality of pins are used to preassemble the plurality of surgical instruments.

9. The surgical kit of item 5, wherein a second size of the second plurality of surgical instruments varies based on a first size of the first plurality of surgical instruments.

10. The surgical kit of claim 1, wherein each of the plurality of trays is an injection-molded tray.

11. The surgical kit of item 1, wherein each of the plurality of trays are 3D-printed.

12. The surgical kit of item 1, wherein each of the plurality of surgical instruments are 3D-printed.

13. A surgical kit for performing a total knee replacement procedure, the surgical kit comprising: a first tray comprising: a plurality of preassembled surgical instruments of a first type, the plurality of preassembled surgical instruments of the first type being used to perform a first step of the total knee replacement procedure; a second tray comprising: a plurality of preassembled surgical instruments of a second type, the plurality of preassembled surgical instruments of the second type being used to perform a second step of the total knee replacement procedure; and a third tray comprising: a plurality of preassembled surgical instruments of a third type, the plurality of preassembled surgical instruments of the third type being used to perform a third step of total knee replacement procedure; wherein the plurality of preassembled surgical instruments of the first type, the plurality of preassembled surgical instruments of the second type, and the plurality of preassembled surgical instruments of the third type are arranged in an order in which they are to be used during the total knee replacement procedure.

14. The surgical kit of item 13, wherein the first tray, the second tray, and the third tray are arranged in a stacked configuration.

15. The surgical kit of item 13, wherein the surgical kit is wrapped in a sterile material.

16. The surgical kit of item 13, wherein each of the first tray, the second tray, and the third tray are each wrapped in a sterile material.

17. The surgical kit of item 13, wherein each of the first tray, the second tray, and the third tray include a plurality of cavities designed to receive the respective plurality of preassembled surgical instruments.

18. A method of forming a surgical kit, the method comprising: forming a plurality of trays, the plurality of trays including a plurality of cavities to receive a plurality of surgical instruments; forming the plurality of surgical instruments; preassembling the plurality of surgical instruments; securing the plurality of preassembled surgical instruments in the plurality of cavities of the plurality of trays; and arranging the plurality of trays in an order in which the plurality of trays are to be used during a surgical procedure.

19. The method of item 18, wherein the method step of forming the plurality of trays further involves 3D-printing the plurality of trays.

20. The method of item 18, wherein the method step of forming the plurality of surgical instruments further involves 3D-printing the plurality of surgical instruments.

21. A method of utilizing a surgical kit comprising: unwrapping the surgical kit to access a plurality of trays, the plurality of trays including a first tray, a second tray, and a third tray; unwrapping the first tray to access a first plurality of preassembled surgical instruments, the first plurality of preassembled surgical instruments having a first type associated with a first step of a surgical procedure; performing a first step of the surgical procedure using the first plurality of preassembled surgical instruments; unwrapping the second tray to access a second plurality of preassembled surgical instruments, the second plurality of preassembled surgical instruments having a second type associated with a second step of the surgical procedure; performing the second step of the surgical procedure using the second plurality of preassembled surgical instruments; unwrapping the third tray to access a third plurality of preassembled surgical instruments, the third plurality of preassembled surgical instruments having a third type associated with a third step in the surgical procedure; and performing the third step in the surgical procedure using the third plurality of preassembled surgical instruments.

22. The method of item 21, further comprising disposing of the first plurality of preassembled surgical instruments, the second plurality of preassembled surgical instruments, and the third plurality of surgical instruments.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A surgical kit, comprising:
a plurality of trays comprising a plurality of surgical instruments made of a disposable material, the plurality of surgical instruments being preassembled in the plurality of trays;
wherein the plurality of surgical instruments are arranged in the plurality of trays in an order in which the plurality of surgical instruments are to be used in a surgical procedure, and the plurality of trays are arranged in the surgical kit in the order in which the plurality of trays are to be used in the surgical procedure.

2. The surgical kit of claim 1, wherein the surgical kit is wrapped in a sterile material.

3. The surgical kit of claim 1, wherein each of the plurality of trays are wrapped in a sterile material.

4. The surgical kit of claim 1, wherein the plurality of trays include at least a first tray and a second tray.

5. The surgical kit of claim 4, wherein the first tray includes a first plurality of surgical instruments of a first type to be used during a first step of the surgical procedure, and the second tray includes a second plurality of surgical instruments of a second type to be used during a second step of the surgical procedure.

6. The surgical kit of claim 5, wherein the plurality of trays further includes a third tray, and the third tray includes a third plurality of surgical instruments of a third type to be used during a third step of the surgical procedure.

7. The surgical kit of claim 1, wherein the plurality of trays each include a plurality of cavities designed to receive the plurality of preassembled surgical instruments.

8. The surgical kit of claim 1, wherein the plurality of surgical instruments further comprise a plurality of components, the plurality of components being coupled together by a plurality of pins to form the plurality of surgical instruments.

9. The surgical kit of claim 5, wherein a second size of the second plurality of surgical instruments varies based on a first size of the first plurality of surgical instruments.

10. The surgical kit of claim 1, wherein each of the plurality of trays is an injection-molded tray.

11. The surgical kit of claim 1, wherein each of the plurality of trays is a 3D-printed tray.

12. The surgical kit of claim 1, wherein each of the plurality of surgical instruments is a 3D-printed surgical instrument.

13. A surgical kit for performing a total knee replacement procedure, the surgical kit comprising:
a first tray comprising a plurality of preassembled surgical instruments of a first type, the plurality of preassembled surgical instruments of the first type being used to perform a first step of the total knee replacement procedure;
a second tray comprising a plurality of preassembled surgical instruments of a second type, the plurality of preassembled surgical instruments of the second type being used to perform a second step of the total knee replacement procedure; and
a third tray comprising a plurality of preassembled surgical instruments of a third type, the plurality of preassembled surgical instruments of the third type being used to perform a third step of total knee replacement procedure;
wherein the plurality of preassembled surgical instruments of the first type, the plurality of preassembled surgical instruments of the second type, and the plurality of preassembled surgical instruments of the third type are arranged in an order in which they are to be used during the total knee replacement procedure.

14. The surgical kit of claim 13, wherein the first tray, the second tray, and the third tray are arranged in a stacked configuration.

15. The surgical kit of claim 13, wherein the surgical kit is wrapped in a sterile material.

16. The surgical kit of claim 13, wherein each of the first tray, the second tray, and the third tray are each wrapped in a sterile material.

17. The surgical kit of claim 13, wherein each of the first tray, the second tray, and the third tray include a plurality of cavities designed to receive the respective plurality of preassembled surgical instruments.

18. A method of forming a surgical kit, the method comprising:
forming a plurality of trays comprising a plurality of cavities to receive a plurality of surgical instruments;
forming the plurality of surgical instruments;
preassembling the plurality of surgical instruments;
securing the plurality of preassembled surgical instruments in the plurality of cavities of the plurality of trays; and
arranging the plurality of trays in an order in which the plurality of trays are to be used during a surgical procedure.

19. The method of claim 18, wherein the method step of forming the plurality of trays comprises 3D printing the plurality of trays.

20. The method of claim 18, wherein the method step of forming the plurality of surgical instruments comprises 3D printing the plurality of surgical instruments.
